(19) United States
(12) Patent Application Publication
(10) Pub. No.: US 2026/0158292 A1
(43) Pub. Date: Jun. 11, 2026

(54) SCANNING OPTICAL FIBER, PHOTOTHERAPY DEVICE, PHOTOTHERAPY SYSTEM, AND PHOTOTHERAPY METHOD

(71) Applicant: OLYMPUS CORPORATION, Tokyo (JP)

(72) Inventor: Takumi HAYASHI, Gunma (JP)

(73) Assignee: OLYMPUS CORPORATION, Tokyo (JP)

(21) Appl. No.: 19/360,135

(22) Filed: Oct. 16, 2025

Related U.S. Application Data
(63) Continuation of application No. PCT/JP2023/015875, filed on Apr. 21, 2023.

(51) Int. Cl. A61N 5/067; A61N 5/01; A61N 5/06; G02B 26/10

(52) U.S. Cl. CPC A61N 5/067; A61N 5/01; G02B 26/103; A61N 2005/063; A61N 2005/0632

(57) ABSTRACT

A scanning optical fiber includes: an optical fiber; and a protruding member that is provided in a distal end region of the optical fiber, and that has an outer diameter larger than an outer diameter of the optical fiber. The protruding member receives a flow of a fluid in a longitudinal direction of the optical fiber toward a distal end of the optical fiber, and generates a force in a radial direction of the optical fiber.

### CROSS-REFERENCE TO RELATED APPLICATIONS

This is a continuation of International Application PCT/JP2023/015875 which is hereby incorporated by reference herein in its entirety.

### TECHNICAL FIELD

The present disclosure relates to a scanning optical fiber, a phototherapy device, a phototherapy system, and a phototherapy method.

### BACKGROUND ART

Known lithotripsy in the related art involves using laser light to fragment a calculus occurring in, for example, a kidney (e.g., see Non Patent Literature 1 and Patent Literature 1). In order to efficiently fragment the calculus, it is preferable to irradiate the calculus with the laser light while scanning the laser light.

Non Patent Literature 1 and Patent Literature 1 each disclose a vibration mechanism for vibrating the distal end of an optical fiber to scan laser light. The vibration mechanism of Non Patent Literature 1 uses a magnetic bead fixed to the optical fiber and a solenoid in the vicinity of the optical fiber, and causes the distal end of the optical fiber to vibrate by means of a magnetic force. The vibration mechanism of Patent Literature 1 uses a plate-shaped operation member disposed in the vicinity of the optical fiber, and causes the distal end of the optical fiber to vibrate due to contraction of a bubble generated at the distal end of the optical fiber by means of the laser light.

### CITATION LIST

### Non Patent Literature

### NPL 1

Layton A. Hall, two others, “Thulium fiber laser stone dusting using an automated, vibrating optical fiber”, Proceedings SPIE 10852, Therapeutics and Diagnostics in Urology 2019, Feb. 26, 2019

### Patent Literature

### PTL 1

PCT International Publication No. WO 2022/190259

### SUMMARY OF DISCLOSURE

An aspect of the present disclosure is a scanning optical fiber including: an optical fiber; and a protruding member that is provided in a distal end region of the optical fiber, and that has an outer diameter larger than an outer diameter of the optical fiber, wherein the protruding member receives a flow of a fluid in a longitudinal direction of the optical fiber toward a distal end of the optical fiber, and generates a force in a radial direction of the optical fiber.

Another aspect of the present disclosure is a phototherapy device including: a medical tube having a channel; an optical fiber inserted into the channel; and a protruding member that is provided in a distal end region of the optical fiber, and that has an outer diameter larger than an outer diameter of the optical fiber, wherein the protruding member receives a flow of a fluid in a longitudinal direction of the optical fiber toward a distal end of the optical fiber, and generates a force in a radial direction of the optical fiber.

Another aspect of the present disclosure is a phototherapy system including: a medical tube having a channel; an optical fiber inserted into the channel; a protruding member that is provided in a distal end region of the optical fiber, and that has an outer diameter larger than an outer diameter of the optical fiber; a fluid supply source that supplies a fluid to the channel; and a laser light source that supplies laser light to the optical fiber, wherein the protruding member receives a flow of a fluid in a longitudinal direction of the optical fiber toward a distal end of the optical fiber, and generates a force in a radial direction of the optical fiber.

Another aspect of the present disclosure is a phototherapy method including: irradiating a target with laser light from a distal end of an optical fiber; and vibrating the distal end of the optical fiber, wherein the vibrating includes generating a flow of a fluid in a longitudinal direction of the optical fiber toward the distal end of the optical fiber, and a protruding member provided in a distal end region of the optical fiber receives the flow of the fluid and generates a force in a radial direction of the optical fiber.

### BRIEF DESCRIPTION OF DRAWINGS

FIG. 1 illustrates the overall configuration of a phototherapy system according to an embodiment of the present disclosure.

FIG. 2 is a partial side view of a scanning optical fiber according to the embodiment.

FIG. 3A is a diagram showing an example of conditions of laser light.

FIG. 3B is a diagram showing another example of the conditions of the laser light.

FIG. 3C is a diagram showing the conditions of the laser light in the case of a conventional scanning optical fiber in which an optical fiber is vibrated due to contraction of a bubble.

FIG. 4 is a flowchart of a phototherapy method according to an embodiment of the present disclosure.

FIG. 5A is a diagram showing the scanning optical fiber during treatment of a calculus.

FIG. 5B is a diagram showing the conventional scanning optical fiber during treatment of a calculus.

FIG. 6 is a cross-sectional view of a wing-shaped operation member.

FIG. 7 is a diagram for explaining the operation of the operation member in FIG. 6.

FIG. 8 is a partial side view of the scanning optical fiber having a columnar or rotary operation member.

FIG. 9A is a diagram showing an example of a cross section of the operation member in FIG. 8.

FIG. 9B is a diagram showing another example of the cross section of the operation member in FIG. 8.

FIG. 9C is a diagram showing another example of the cross section of the operation member in FIG. 8.

FIG. 9D is a diagram showing another example of the cross section of the operation member in FIG. 8.

FIG. 9E is a diagram showing another example of the cross section of the operation member in FIG. 8.

FIG. 9F is a diagram showing another example of the cross section of the operation member in FIG. 8.

FIG. 9G is a diagram showing another example of the cross section of the operation member in FIG. 8.

### DESCRIPTION OF EMBODIMENT

A scanning optical fiber, a phototherapy device, a phototherapy system, and a phototherapy method according to an embodiment of the present disclosure will be described below with reference to the drawings.

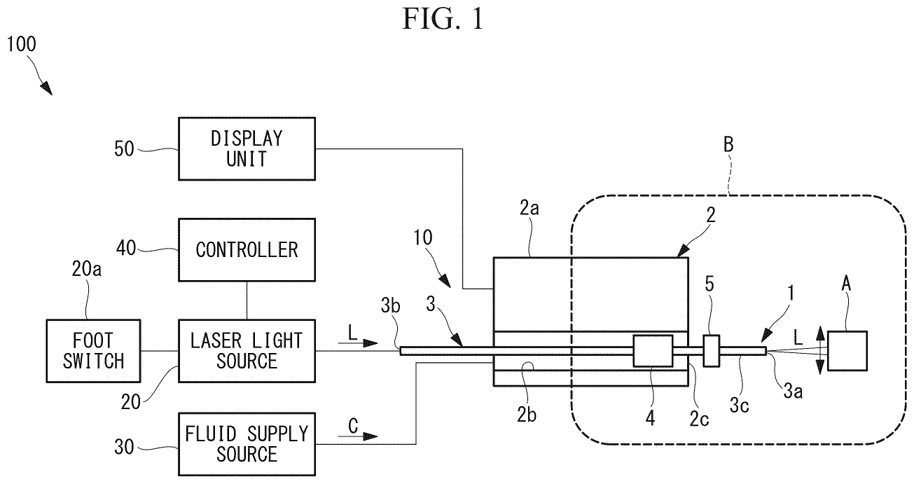

As shown in FIG. 1, a phototherapy system 100 according to this embodiment is a lithotripsy system that uses laser light L to fragment a calculus serving as a treatment target A. The phototherapy system 100 includes a phototherapy device 10, a laser light source 20, a fluid supply source 30, and a controller 40.

The phototherapy device 10 includes a scanning optical fiber 1 and a medical tube 2.

The medical tube 2 is an endoscope having an elongated flexible insertion portion 2a. An image of the interior of a body B, which is acquired by the endoscope 2, may be displayed on a display unit 50. The endoscope 2 has a channel 2b that penetrates the insertion portion 2a in the longitudinal direction, and the channel 2b has, at the distal end thereof, an outlet 2c that opens to a distal end surface of the endoscope 2. The channel 2b of the endoscope 2 is for a perfusate C and an optical fiber 3, and also serves as a passage through which another treatment tool is inserted.

The scanning optical fiber 1 includes the optical fiber 3, a fiber retaining portion 4, and an operation member (protruding member) 5.

The optical fiber 3 has a distal end 3a that outputs the laser light L and a proximal end 3b that is connected to the laser light source 20. The optical fiber 3 has an outer diameter smaller than the inner diameter of the channel 2b and is insertable into the channel 2b.

The fiber retaining portion 4 retains a portion of the optical fiber 3 at a position spaced apart from the distal end 3a in the longitudinal direction, and fixes the portion of the optical fiber 3 so as not to move in the radial direction within the channel 2b. For example, the fiber retaining portion 4 is a tubular member attached to a side surface of the portion of the optical fiber 3, and has an outer diameter slightly smaller than the inner diameter of the channel 2b.

A cantilever-like distal end region 3c of the optical fiber 3, which protrudes from a distal end surface of the retaining portion 4, is a vibration region that vibrates in the radial direction. The vibration region 3c vibrates in the radial direction with the portion retained by the retaining portion 4 serving as a fulcrum, whereby the laser light L output from the distal end 3a is scanned.

In addition, the fiber retaining portion 4 has a shape that allows the perfusate C to pass through the fiber retaining portion 4 in the longitudinal direction, and has, for example, a flow path extending in the longitudinal direction.

The operation member 5 is fixed to the side surface of a portion of the vibration region 3c between the distal end 3a and the fiber retaining portion 4. The distal end of the operation member 5 is disposed at a position spaced apart from the distal end 3a to the proximal end side, and the proximal end of the operation member 5 is disposed at a position spaced apart from the distal end of the fiber retaining portion 4 to the distal end side.

The operation member 5 has an outer diameter larger than the outer diameter of the optical fiber 3 and radially protrudes from the side surface of the optical fiber 3. The maximum outer diameter of the operation member 5 is equal to or less than the inner diameter of the channel 2b, and the operation member 5 can pass through the channel 2b together with the optical fiber 3. The outer diameter of the operation member 5 is the dimension of the operation member 5 in a direction orthogonal to a longitudinal axis 3d of the optical fiber 3. In one example, the inner diameter of the channel 2b is 1.2 mm, and the maximum outer diameter of the operation member 5 is 1.2 mm or less.

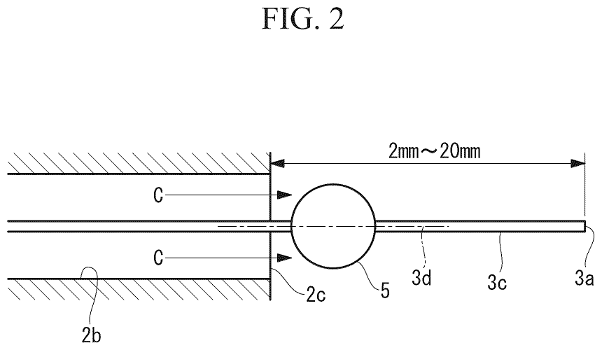

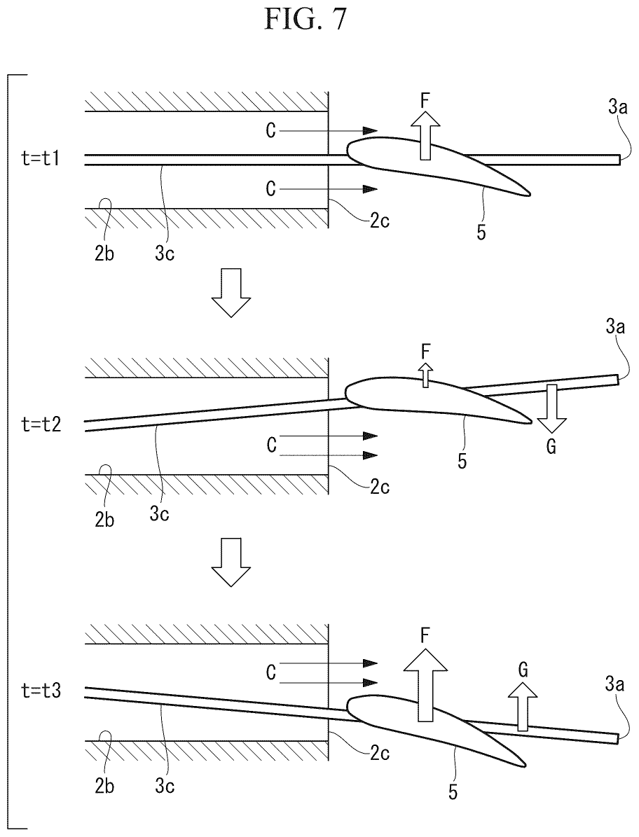

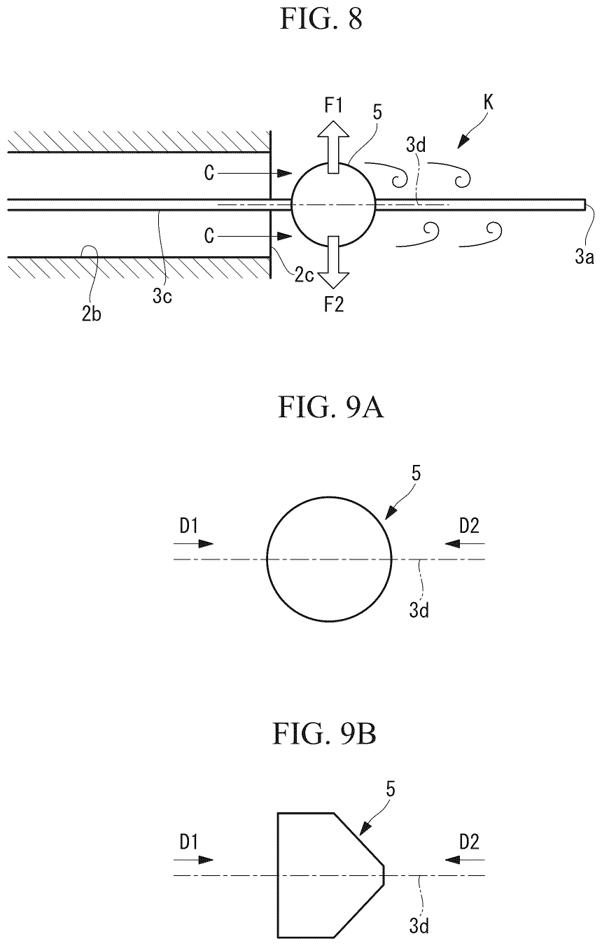

As shown in FIG. 2, the perfusate C passing through the channel 2b from the proximal end toward the distal end forms, in the vicinity of the outlet 2c, a flow parallel or substantially parallel to the longitudinal direction of the optical fiber 3 toward the distal end 3a. The operation member 5 is disposed at such a position that the operation member 5 blocks the flow of the perfusate C, receives the flow of the perfusate C, and generates a lift force F in the radial direction of the optical fiber 3 (see FIGS. 7 and 8). Specifically, the operation member 5 deflects the flow direction of the perfusate C in the radial direction, thereby generating a lift force F perpendicular to the flow direction of the perfusate C. In the vibration region 3c displaced in the radial direction while being deflected according to the lift force F, a restoring force G for enabling the vibration region 3c to return to the linear shape is generated in the radial direction (see FIG. 7). The operation member 5 generates the lift force F such that the direction of the resultant force between the lift force F and the restoring force G is periodically inverted, thereby causing the vibration region 3c to vibrate. The specific configuration of the operation member 5 will be described in detail later.

In order to prevent the operation member 5 from interfering with the inner surface of the channel 2b, it is preferable that at least a portion of the operation member 5 be disposed outside the outlet 2c, and it is more preferable that the entire operation member 5 be disposed outside the outlet 2c. The preferable protrusion length of the optical fiber 3 from the distal end of the endoscope 2 to the distal end 3a is 2 to 20 mm. Therefore, the proximal end of the operation member 5 is preferably disposed at a position 2 to 20 mm from the distal end 3a.

The laser light source 20 is, for example, a laser oscillator and is optically connected to the proximal end 3b of the optical fiber 3. In response to an operation performed on a foot switch 20a, the laser light source 20 outputs pulsed laser light L for treating the target A. The laser light L is, for example, infrared light.

The fluid supply source 30 is fluidically connected to the proximal end of the channel 2b and supplies the perfusate C, such as a physiological saline solution, to the channel 2b. For example, the fluid supply source 30 has a bag for accommodating the perfusate C and a tube that connects the bag and the channel 2b, and supplies the perfusate C to the channel 2b by natural dripping from the bag.

In order to stably generate the lift force F by means of the operation member 5, it is desirable that the perfusate C form a uniform flow in the vicinity of the outlet 2c. Therefore, the fluid supply source 30 supplies the perfusate C at a constant flow rate (for example, 20 ml/min).

The magnitude of the lift force F generated by the operation member 5 depends on the flow rate of the perfusate C. In order to enable the magnitude of the lift force F to be adjusted, the fluid supply source 30 may be capable of changing the flow rate of the perfusate C and may have, for example, a pump capable of controlling the flow rate.

The fluid supply source 30 may temporally change the flow rate of the perfusate C in synchronization with the vibration of the optical fiber 3.

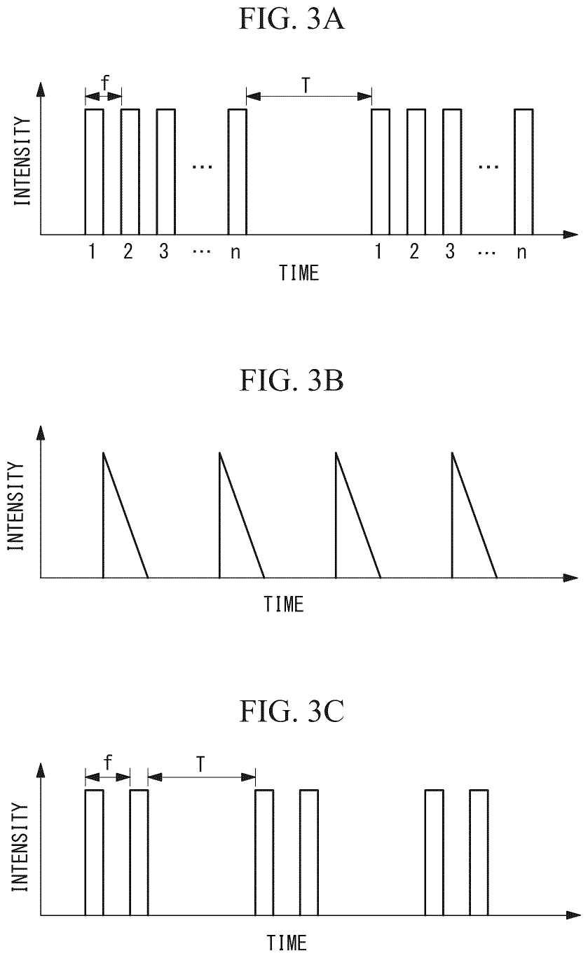

The controller 40 controls the conditions of the laser light L output by the laser light source 20. FIGS. 3A and 3B show examples of the laser light L. The conditions include, for example, the pulse waveform, the pulse number n in one pulse group, the repetition frequency f, an interval T between the pulse groups, etc.

Next, a phototherapy method using the scanning optical fiber 1, the phototherapy device 10, and the phototherapy system 100 will be described.

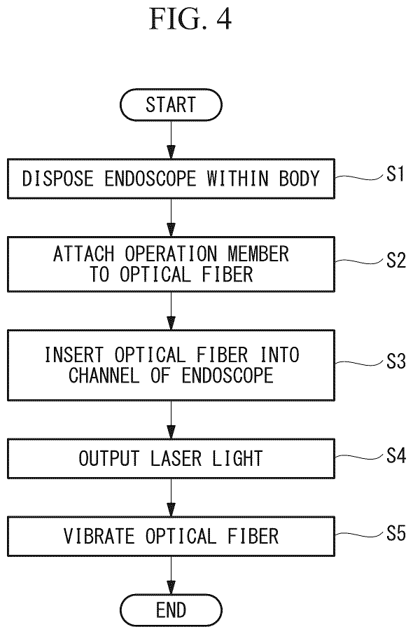

As shown in FIG. 4, the phototherapy method includes: step S1 for disposing the endoscope 2 within the body B; step S2 for preparing the optical fiber 3 to which the operation member 5 is attached; step S3 for inserting the optical fiber 3 to which the operation member 5 is attached into the channel 2b of the endoscope 2; step S4 for irradiating the target A with the laser light L; and step S5 for vibrating the distal end 3a of the optical fiber 3.

An operator, such as a surgeon, inserts the endoscope 2 into, for example, the kidney through the urethra (step S1).

Next, the operator inserts the optical fiber 3 into, for example, through-holes respectively provided in the retaining portion 4 and the operation member 5, thereby attaching the retaining portion 4 and the operation member 5 to the side surface of the optical fiber 3 (step S2). The optical fiber 3 to which the retaining portion 4 and the operation member 5 are attached in advance may be provided to the operator.

Next, the operator inserts the optical fiber 3 to which the retaining portion 4 and the operation member 5 are attached into the channel 2b, disposes the distal end 3a of the optical fiber 3 and the operation member 5 outside the channel 2b through the outlet 2c, and disposes the retaining portion 4 inside the channel 2b (step S3).

Next, the operator steps on the foot switch 20a to cause the laser light source 20 to start outputting the laser light L (step S4). The laser light L is radiated onto the target A from the distal end 3a of the optical fiber 3, thereby fragmenting a calculus which is the target A.

Step S5 is performed concurrently with step S4. In step S5, the operator supplies the perfusate C to the channel 2b from the fluid supply source 30, thereby generating, in the vicinity of the outlet 2c and in the periphery of the vibration region 3c, a flow of the perfusate C in the longitudinal direction of the vibration region 3c toward the distal end 3a (step S5). As a result of the flow of the perfusate C colliding with the operation member 5, the lift force F is generated, and thus causing the vibration region 3c to vibrate. By doing so, the laser light L is scanned on the calculus A, so that the calculus A is irradiated over a wide range. Therefore, it is possible to enhance the treatment efficiency using the laser light L, for example, the efficiency in fragmenting the calculus A.

In addition, in step S5, the perfusate C improves a visual field defect of the endoscope 2 due to crushed pieces of the calculus A, whereby a clear visual field is obtained, and also suppresses a rise in the intrarenal temperature due to the laser light L.

As described above, during treatment of the calculus A, the perfusate C is supplied into the body B through the channel 2b in order to improve a visual field defect and to suppress a rise in the intrarenal temperature. With this embodiment, the energy of the flow of the perfusate C is converted into the lift force F in the radial direction of the optical fiber 3 by means of the operation member 5, and the lift force F causes the optical fiber 3 to vibrate. In other words, the flow of the perfusate C is used as a drive source for the vibration. Therefore, a drive source such as a power source is not required, and it is possible to realize a compact device 10 and system 100.

In addition, in a case in which a vibration mechanism for generating an electromagnetic field is used, as in Non Patent Literature 1, the electromagnetic field may affect the quality of an endoscopic image. Since the scanning optical fiber 1 of this embodiment does not require an electromagnetic field, a good endoscopic image can be obtained.

In addition, since the vibration of the optical fiber 3 caused by the lift force F does not depend on the conditions of the laser light L, it is possible to arbitrarily set the conditions of the laser light. Therefore, as shown in FIGS. 3A and 3B, it is possible to use the laser light L having conditions corresponding to the size, type, etc. of the target A, thereby further enhancing the treatment effect on the target A.

In a case in which the distal end 3a is vibrated by utilizing contraction of a bubble formed at the distal end 3a by means of the laser light L, as in Patent Literature 1, it is necessary to use the laser light L having specific conditions. For example, as shown in FIG. 3C, the pulse number n, repetition frequency f, and interval T are limited to prescribed ranges. Therefore, it is difficult to adjust the conditions of the laser light L depending on the target A.

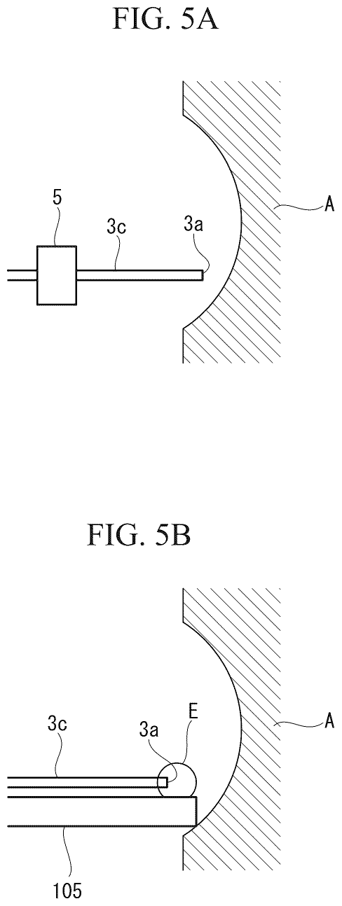

In addition, with this embodiment, the operation member 5 is disposed farther on the proximal end side than the distal end 3a, and the scanning optical fiber 1 does not have a structure in the vicinity of the distal end 3a. Therefore, as shown in FIG. 5A, it is possible to prevent the scanning of the laser light L from being disturbed, for example, as a result of the operation member 5 interfering with the calculus A.

In a case in which a plate-shaped operation member 105 that causes a contraction force of a bubble E to act on the distal end 3a is provided, as in Patent Literature 1, the scanning of the laser light L may be disturbed, for example, as a result of the operation member 105 interfering with the calculus A (see FIG. 5B).

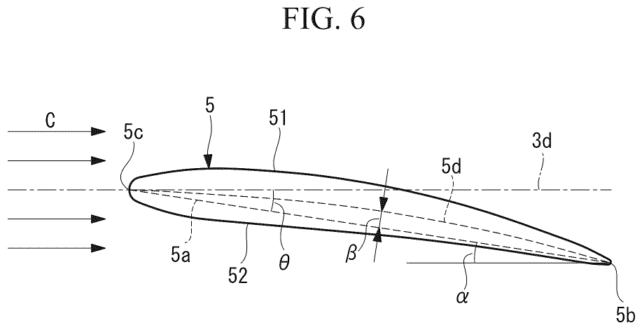

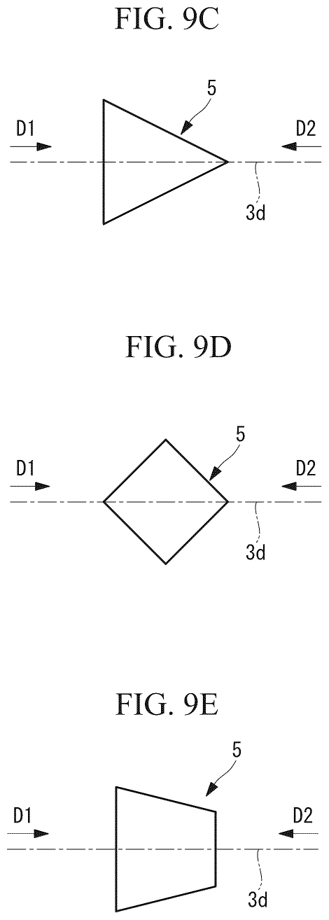

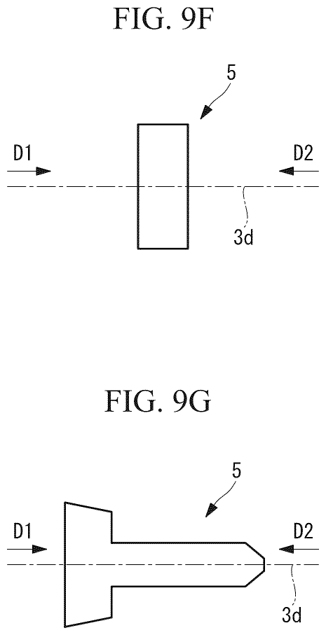

Next, specific examples of the operation member 5 will be described. FIGS. 6 and 7 show a plate-shaped operation member 5, and FIGS. 8 to 9G show a columnar or rotary operation member 5.

The operation member 5 in FIG. 6 has a first surface 51 and a second surface 52 facing each other in the thickness direction, and is arranged so as to be inclined with respect to the longitudinal axis 3d of the optical fiber 3. A cross section of the operation member 5 in a direction along the longitudinal axis 3d has a wing shape. The wing shape is a shape in which a lift force is generated by the difference between the pressure on the first surface 51 side and the pressure on the second surface 52 side, and is generally a streamline shape having a sharp trailing edge 5b and a round leading edge 5c. A chord 5a of the operation member 5 is inclined at an angle θ with respect to the longitudinal axis 3d, and forms an elevation angle α with the flow direction of the perfusate C. When the vibration region 3c is disposed at an initial position without deflection, the elevation angle α is equal to the angle θ.

As long as the lift force F of the magnitude required for the vibration of the vibration region 3c is obtained, the plate-shaped operation member 5 may have a cross section of another shape. For example, the operation member 5 may be a flat plate that is inclined with respect to the longitudinal axis 3d and deflects the flow of the perfusate C. The flat plate-shaped operation member 5 receives a reaction force from the flow of the perfusate C by deflecting the flow, and generates the lift force F due to the reaction force.

FIG. 7 illustrates the vibration of the vibration region 3c caused by the lift force F and the restoring force G of the optical fiber 3.

When the vibration region 3c is disposed at the initial position, the flow of the perfusate C causes the operation member 5 to generate the lift force F (t=t1). The restoring force G of the vibration region 3c is zero at the initial position, and the vibration region 3c is displaced in the radial direction while being deflected according to the lift force F.

As a result of the vibration region 3c being displaced from the initial position, the restoring force G is generated in the opposite direction from the lift force F. As the displacement increases, the restoring force G increases. In addition, as the displacement increases, the elevation angle α decreases, thereby decreasing the lift force F. Therefore, the direction of the resultant force between the lift force F and the restoring force G is inverted, and the vibration region 3c is subsequently displaced in the opposite direction toward the initial position (t=t2).

In the process of the displacement in the opposite direction toward the initial position, the lift force F increases and the restoring force G decreases. Therefore, the direction of the resultant force is inverted again.

In a state in which the vibration region 3c is displaced from the initial position in the opposite direction, the direction of the restoring force G is the same as the direction of the lift force F (t=t3). As the displacement increases, the restoring force G increases. In addition, as the displacement increases, the elevation angle α increases, thereby increasing the lift force F. Therefore, the vibration region 3c is subsequently displaced toward the initial position.

As described above, the wing-shaped operation member 5 always generates the lift force F in the same direction, and the magnitude of the lift force F changes in accordance with the elevation angle α. In addition, the direction of the restoring force G periodically changes. Therefore, the direction of the resultant force is periodically inverted, and this enables the vibration region 3c to vibrate.

The lift force F generated by the operation member 5 depends on the angle θ and the camber β. The camber β is a distance between the chord 5a and a center line 5d. The angle θ and the camber β are designed so as to achieve the vibration as described above.

The columnar or rotary operation member 5 in FIG. 8 is plane-symmetric with respect to a plane including the longitudinal axis 3d, and generates Karman vortex K on the distal end side of the operation member 5. The Karman vortex K refers to a plurality of rows of vortices, and the swirling direction of the vortices is alternately reversed. Therefore, the operation member 5 alternately generates a lift force F1 and a lift force F2 in the opposite direction from the lift force F1, thereby causing the vibration region 3c to vibrate. In a case of the columnar operation member 5, the distal end 3a vibrates one-dimensionally in the radial direction. In a case of the rotary operation member 5, the distal end 3a vibrates two-dimensionally within a plane in the radial direction.

The maximum outer diameter of the operation member 5 is equal to or less than the inner diameter (for example, 1.2 mm) of the channel 2b so that the operation member 5 can pass through the channel 2b.

The columnar body has a height orthogonal to the longitudinal axis 3d and a pair of bases disposed on both sides of the longitudinal axis 3d. FIGS. 9A to 9G show examples of the shape of the bases or a cross section perpendicular to the height, and the height is a direction orthogonal to the paper surface. As shown in each of FIGS. 9A to 9G, the bases or the cross section may have a circular shape, bullet shape, triangular shape, square shape, trapezoidal shape, rectangular shape having short sides in a direction along the longitudinal axis 3d, or substantially T-shape. The bases and the cross section may have another polygonal shape or other shape that is line-symmetric with respect to the longitudinal axis 3d as long as the Karman vortex K can be generated.

The rotary body is a three-dimensional body in which a plane figure that is line-symmetric with respect to the longitudinal axis 3d, as shown in FIGS. 9A to 9G, is rotated about the longitudinal axis 3d, and is symmetric with respect to the longitudinal axis 3d. For example, the rotary operation member 5 is a sphere (FIG. 9A). As described above, the rotary operation member 5 causes the distal end 3a to vibrate two-dimensionally; thus, there is an advantage in that it is possible to radiate the laser light L over a wider range.

The cross section of the columnar body and the rotary body may have a shape in which the plane figure in any one of FIGS. 9A to 9G is inverted in the left-right direction. In other words, the flow direction of the perfusate C with respect to the operation member 5 may be a direction D1 from the left to the right, or may be a direction D2 from the right to the left.

As is well known, the columnar body and the rotary body generate Karman vortex when the Reynolds number is within a prescribed range, and the Reynolds number depends on the flow rate and the dimensions of the columnar body or the rotary body. Therefore, the dimensions of the operation member 5 are designed so as to generate the Karman vortex at a desired flow rate of the perfusate C.

With respect to the columnar operation member 5, the vibration direction of the vibration region 3c depends on the height of the operation member 5.

In description of a cylindrical operation member 5, in a case in which the height is sufficiently large (for example, the height is larger than the diameter of the bases), the vibration direction is a direction parallel to the bases (vertical direction in FIG. 9A). Meanwhile, as the height decreases, the shape of the operation member 5 becomes closer to a disk. In a case in which the height is sufficiently small (for example, the height is approximately 1/10 of the diameter of the bases), the vibration direction is the height direction (direction perpendicular to the paper surface in FIG. 9A).

As described above, the vibration direction approaches the height direction as the height decreases and the operation member 5 has a shape closer to a plate. Similarly in a case of the operation member 5 having the bases of another shape, the vibration direction depends on the height.

Although the endoscope 2 serves as a medical tube in the abovementioned embodiment, the medical tube may be any elongated medical device having a channel 2b, and may be, for example, a catheter.

Although the fiber retaining portion 4 is attached to the side surface of the optical fiber 3 in the abovementioned embodiment, alternatively, the fiber retaining portion 4 may be provided on the inner surface of the channel 2b.

Although the scanning optical fiber 1 is inserted into the body B through the channel 2b of the medical tube in the abovementioned embodiment, alternatively, the scanning optical fiber 1 may be inserted into the body B independently of the medical tube. In this case, the outer diameter of the operation member 5 may be larger than the inner diameter of the channel 2b. In addition, the flow of the perfusate C required to generate the lift force F may be generated in the periphery of the vibration region 3c by utilizing an arbitrary means.

Although the embodiment of the present disclosure and the modifications thereof have been described above, the present disclosure is not limited thereto, and are modifiable, as appropriate, within a range not departing from the scope of the present disclosure.

For example, the phototherapy device 10 and the phototherapy system 100 are not limited to lithotripsy and are applicable to any treatment involving irradiating a target A with light. In particular, the phototherapy device 10 and the phototherapy system 100 may be suitably applied to treatment performed while supplying a liquid or gas. The fluid supplied to the channel 2b by the fluid supply source 30 is also appropriately selected depending on the type of treatment. Specifically, the fluid supply source 30 may supply a different liquid or gas to the channel 2b.

In addition, the scanning optical fiber 1 may be utilized not only for treatment but also for other uses involving scanning laser light.

### REFERENCE SIGNS LIST

1 scanning optical fiber
2 endoscope (medical tube)
3 optical fiber
3c vibration region (distal end region)
3d longitudinal axis
5 operation member (protruding member)
10 phototherapy device
20 laser light source
30 fluid supply source
100 phototherapy system
A target
C perfusate (fluid)
F, F1, F2 lift force
L laser light

1. A scanning optical fiber comprising:
an optical fiber; and
a protruding member that is provided in a distal end region of the optical fiber, and that has an outer diameter larger than an outer diameter of the optical fiber,
wherein the protruding member is configured to receive a flow of a fluid in a longitudinal direction of the optical fiber toward a distal end of the optical fiber, and generate a force in a radial direction of the optical fiber.

2. The scanning optical fiber according to claim 1, wherein the protruding member is a plate-shaped member that is inclined with respect to a longitudinal axis of the optical fiber.

3. The scanning optical fiber according to claim 2, wherein the protruding member has a wing-shaped cross section.

4. The scanning optical fiber according to claim 1, wherein:
the protruding member is a columnar body or a rotary body;
the columnar body has a height in a direction orthogonal to a longitudinal axis of the optical fiber, and is plane-symmetric with respect to a plane including the longitudinal axis; and
the rotary body is symmetric with respect to the longitudinal axis.

5. The scanning optical fiber according to claim 4, wherein the protruding member has a circular, polygonal, or bullet-shaped cross section that is line-symmetric with respect to the longitudinal axis.

6. The scanning optical fiber according to claim 1, wherein the protruding member has the outer diameter equal to or less than an inner diameter of a channel of a medical tube into which the scanning optical fiber is inserted.

7. A phototherapy device comprising:
a medical tube having a channel;
an optical fiber inserted into the channel; and
a protruding member that is provided in a distal end region of the optical fiber, and that has an outer diameter larger than an outer diameter of the optical fiber,
wherein the protruding member receives a flow of a fluid in a longitudinal direction of the optical fiber toward a distal end of the optical fiber, and generates a force in a radial direction of the optical fiber.

8. The phototherapy device according to claim 7, wherein the outer diameter of the protruding member is equal to or less than an inner diameter of the channel.

9. A phototherapy system comprising:
a medical tube having a channel;
an optical fiber inserted into the channel;
a protruding member that is provided in a distal end region of the optical fiber, and that has an outer diameter larger than an outer diameter of the optical fiber;
a fluid supply source that supplies a fluid to the channel; and
a laser light source that supplies laser light to the optical fiber,
wherein the protruding member receives a flow of a fluid in a longitudinal direction of the optical fiber toward a distal end of the optical fiber, and generates a force in a radial direction of the optical fiber.

10. The phototherapy system according to claim 9, wherein the protruding member is a plate-shaped member that is inclined with respect to a longitudinal axis of the optical fiber.

11. The phototherapy system according to claim 10, wherein the protruding member has a wing-shaped cross section.

12. The phototherapy system according to claim 9, wherein:
the protruding member is a columnar body or a rotary body;
the columnar body has a height in a direction orthogonal to a longitudinal axis of the optical fiber, and is plane-symmetric with respect to a plane including the longitudinal axis; and
the rotary body is symmetric with respect to the longitudinal axis.

13. The phototherapy system according to claim 12, wherein the protruding member has a circular, polygonal, or bullet-shaped cross section that is line-symmetric with respect to the longitudinal axis.

14. The phototherapy system according to claim 9, wherein the protruding member has the outer diameter equal to or less than an inner diameter of the channel of the medical tube into which the optical fiber is inserted.

15. A phototherapy method comprising:
irradiating a target with laser light from a distal end of an optical fiber; and
vibrating the distal end of the optical fiber,
wherein the vibrating includes generating a flow of a fluid in a longitudinal direction of the optical fiber toward the distal end of the optical fiber, and a protruding member provided in a distal end region of the optical fiber receives the flow of the fluid and generates a force in a radial direction of the optical fiber.

16. The phototherapy method according to claim 15, wherein the protruding member is a plate-shaped member that is inclined with respect to a longitudinal axis of the optical fiber.

17. The phototherapy method according to claim 16, wherein the protruding member has a wing-shaped cross section.

18. The phototherapy method according to claim 15, wherein:
the protruding member is a columnar body or a rotary body;
the columnar body has a height in a direction orthogonal to a longitudinal axis of the optical fiber, and is plane-symmetric with respect to a plane including the longitudinal axis; and
the rotary body is symmetric with respect to the longitudinal axis.

19. The phototherapy method according to claim 18, wherein the protruding member has a circular, polygonal, or bullet-shaped cross section that is line-symmetric with respect to the longitudinal axis.

20. The phototherapy method according to claim 15, wherein the protruding member has an outer diameter equal to or less than an inner diameter of a channel of a medical tube into which the optical fiber is inserted.
